(19) United States
(12) Patent Application Publication
(10) Pub. No.: US 2024/0302374 A1
(43) Pub. Date: Sep. 12, 2024

(54) SPECTROGRAPHIC DETECTION OF ACUTE LEUKEMIA

(71) Applicant: KING SAUD UNIVERSITY, RIYADH (SA)

(72) Inventors: MOHAMAD SALEH ALSALHI, RIYADH (SA); SANDHANASAMY DEVANESAN, RIYADH (SA); FATMA S. AL-QAHTANI, RIYADH (SA); VADIVEL MASILAMANI, RIYADH (SA)

(21) Appl. No.: 18/180,628

(22) Filed: Mar. 8, 2023

(51) Int. Cl. G01N 33/574; G01N 21/64

(52) U.S. Cl. CPC G01N 33/57426; G01N 21/6428; G01N 2021/6417

(57) ABSTRACT

The spectrographic detection of acute leukemia provides for the early detection of acute leukemia, distinguishing between normal blood and blood infected by either Acute Lymphoblastic Leukemia (ALL) or blood infected by Acute Myeloid Leukemia (AML). The spectrographic detection of acute leukemia also provides an alternative method for monitoring the progress of treatment for acute leukemia. The method uses fluorescence spectroscopy techniques, including fluorescence emission spectra (FES) of acetone extracts of red blood cells, synchronous fluorescence emission spectra (SFES) and synchronous fluorescence excitation spectra (SFXS) of different biomolecules found in blood plasma, and comparing ratios of the intensities obtained at certain wavelength peaks.

### BACKGROUND

### 1. Field

The disclosure of the present patent application relates to early detection and follow-up monitoring of acute leukemia, and particularly to the spectrographic detection of acute leukemia using fluorescence spectroscopy techniques.

### 2. Description of the Related Art

Blood cells are formed in the bone marrow, which is the spongy tissue found inside the bones. Blood-forming stem cells divide to produce either more stem cells or immature cells that become mature blood cells over time. In leukemia, the process goes out of control and the cells continue to divide but not mature. These immature aberrant cells become malignant.

There are two major classifications in leukemia, including (1) Acute Lymphoblastic Leukemia (ALL), which is the cancer of lymphoid line of blood cell (i.e., there is rapid growth of lymphoblastic immature cells); and (2) Acute Myeloid Leukemia (AML), which is the cancer of the myeloid line of blood cells. These two classes of leukemia are aggressive, virulent, malignancies and may be fatal within weeks or months, if the subjects go undetected and treated.

Chronic Lymphoblastic Leukemia (CLL) and Chronic Myeloid Leukemia (CML) are chronic, slow-growing malignancies of the above acute leukemia. Only 5% of those diagnosed with CLL or CML, left untreated, will end up developing into ALL or AML.

A lymphoid stem cell becomes a lymphoblast cell and then one of three types of lymphocytes (white blood cells): B lymphocytes that make antibodies to help fight infection; T lymphocytes that help B lymphocytes make the antibodies that help fight infection; and natural killer cells that attack cancer cells and viruses. A myeloid stem cell becomes one of three types of mature blood cells: red blood cells that carry oxygen to all tissues of the body; platelets that form blood clots to stop bleeding; and granulocytes (white blood cells) that fight infection and disease. Normal white blood cells (WBC) grow, do their function and die, but these abnormal blasts grow and keep growing. Hence, when these abnormal blasts become too many, normal WBCs get overwhelmed, choked and stifled.

There are no specific signs or symptoms that would allow a diagnosis of AL to be made. The most common signs and symptoms are caused by the bone marrow being unable to produce enough normal blood cells. These signs and symptoms include anemia due to lack of red blood cells; weakness, tiredness, shortness of breath, light-headedness, and palpitations; infections due to lack of normal white blood cells; infections are more frequent, more severe, and last longer; fever, malaise (general feeling of illness) and sweats; purpura (small bruises in skin), nosebleeds, bleeding gums; and bleeding and bruising due to lack of platelets.

The standard procedure of diagnosis is extensive blood count analysis and bone marrow biopsy. Once leukemia is confirmed, the standard regimen of treatment consists mostly of chemotherapy, occasional radiotherapy, and bone marrow transplantation. The five-year survival rate is 57% in USA, 20 to 40% in most other countries.

Leukemia symptoms are vague, not specific, and often confused with many other diseases. And as has been specifically warned by a report by the Mayo Clinic, early symptoms of leukemia are often overbooked and confused with flu. Further, it is not detected by routine blood test, but only by specific blood tests, and is confirmed by bone marrow biopsy, X-ray, and other scans. It is important to mention that there is no staging for leukemia, as in the case of cancer of the breast; because staging is related to the physical extension of the organ affected by malignancy. In the case of leukemia, it occurs all over the body. However, unmistakable diagnosis, as soon as possible, is the key for complete and/or delayed remission.

There is a need for a method or technique for diagnosing acute leukemia as early as possible and monitoring the course of treatment that is substantially reliable, quick, low cost, and capable of being performed on existing equipment, even in small clinics.

Thus, the spectrographic detection of acute leukemia solving the aforementioned problems is desired.

### SUMMARY

The spectrographic detection of acute leukemia provides for the early detection of acute leukemia, distinguishing between normal blood and blood infected by either Acute Lymphoblastic Leukemia (ALL) or blood infected by Acute Myeloid Leukemia (AML). The spectrographic detection of acute leukemia also provides an alternative method for monitoring the progress of treatment for acute leukemia. The method uses fluorescence spectroscopy techniques, including fluorescence emission spectra (FES) of acetone extracts of red blood cells, synchronous fluorescence emission spectra (SFES) and synchronous fluorescence excitation spectra (SFXS) of different biomolecules found in blood plasma, and comparing ratios of the intensities obtained at certain wavelength peaks.

In the method, a blood sample is collected from a patient exhibiting symptoms of a blood disorder. If the volume of plasma is 1.7 to 2.3 times the volume of cellular components, the patient may have some form of acute leukemia. An FES spectrum of an acetone extract of the blood sample is taken, and if the ratio of intensities at 630 nm:585 nm is 0.5-0.7, the patient may have anemia or acute leukemia. An SFXS spectrum of the sample is taken, and if the peak intensity is around 70, the blood is normal, but if the peak intensity is between 15-30, either ALL or AML is present. An FES spectrum of the samples is taken, and if the ratio of intensities at 450 nm:370 nm is between 1-2, the blood is normal, but above 3, either ALL or AML is present. An SFXS spectrum of the sample is then taken, and if the ratio of intensities at 270 nm:300 nm is 0.8 and the ratio of intensities at 450 nm:370 nm is 2 to 4, AML is present; otherwise ALL is present.

In order to prevent a false positive due to the presence of beta thalassemia, an SFES spectrum of the blood sample may be taken. If the ratio of intensities at 555 nm:460 nm is greater than 4, beta thalassemia is present, but if less than 1.5, acute leukemia is present.

These and other features of the present subject matter will become readily apparent upon further review of the following specification.

### BRIEF DESCRIPTION OF THE DRAWINGS

FIG. 1A is a fluorescence emission spectroscopy (FES) spectrum of normal blood.

FIG. 1B is an FES spectrum of blood showing acute lymphoblastic leukemia (ALL).

FIG. 1C is an FES spectrum of blood showing acute myeloid leukemia (AML).

FIG. 2 is a composite synchronous fluorescence excitation spectrum (SFXS) comparing normal blood, ALL blood, and AML blood.

FIG. 3 is a composite FES spectrum comparing plasma for normal blood with plasma for acute leukemia (either ALL or AML).

FIG. 4 is a composite synchronous fluorescence emission spectrum (SFES) comparing ALL blood with beta-thalassemia blood.

Similar reference characters denote corresponding features consistently throughout the attached drawings.

### DETAILED DESCRIPTION OF THE PREFERRED EMBODIMENTS

The spectrographic detection of acute leukemia uses a spectrofluorometer, which measures the fluorescence intensities of a set of bio-molecules and quantifies their relative concentrations in the blood components of the acute leukemia (AL) patients in comparison with the age-adjusted normal healthy controls.

The present disclosure is about spectroscopic discrimination of acute lymphoblastic leukemia (ALL) and acute myeloid leukemia (AML) and discrimination of the above two from the normal control.

The instrument used was a conventional spectrofluorometer, such as Perkin Elmer (LS 50 or 55) with facility for acquiring fluorescence emission spectra (FES) of acetone extract of red blood cells (RBC) of subjects in the range of 425-700 nm when the samples were excited by an incident light of 400 nm, with a spectral width of 10 nm (all obtainable with an Xe lamp as a source and a combination of grating and a slit for wavelength selection).

The same instrument is capable of obtaining synchronous fluorescence emission spectra (SFES) of different bio-molecules of blood plasma (such as tyrosine, tryptophan, etc.) in the wavelength range of 200-700 nm. This is done by keeping an offset of 10 nm between the excitation and emission gratings. If instead, the offset between the above two gratings is fixed at 70 nm, the synchronous fluorescence excitation spectra (SFXS) of different bio-molecules of blood plasma (such as tyrosine, tryptophan, etc.) is obtained.

In general, 5 ml blood was collected, e.g., from each subject in an EDTA violet vial, which contained standard anticoagulant. The subjects, normal control, and patients were informed about the investigation, and proper consents were obtained. The Institutional Review Board approval [E-17 2267 dt 27 march 17] had been obtained for this investigation from KKUH of KSU of Riyadh, KSA.

The violet vial is rocked five times gently and centrifuged for 15 minutes at 3000 rpm to separate out plasma from the cellular components. At the end of 15 minutes, the top supernatant greenish yellow liquid, plasma, is pipetted out and subjected to (SFES) and (SFXS) analyses. The bottom thick, semi-solid paste of blood containing mostly RBC is lysed with acetone (1:2 v:v) and then centrifuged to obtain a clear, transparent supernatant. This consisted mostly of the fluorescent bio-molecules of RBC and is pipetted out and subjected to FES.

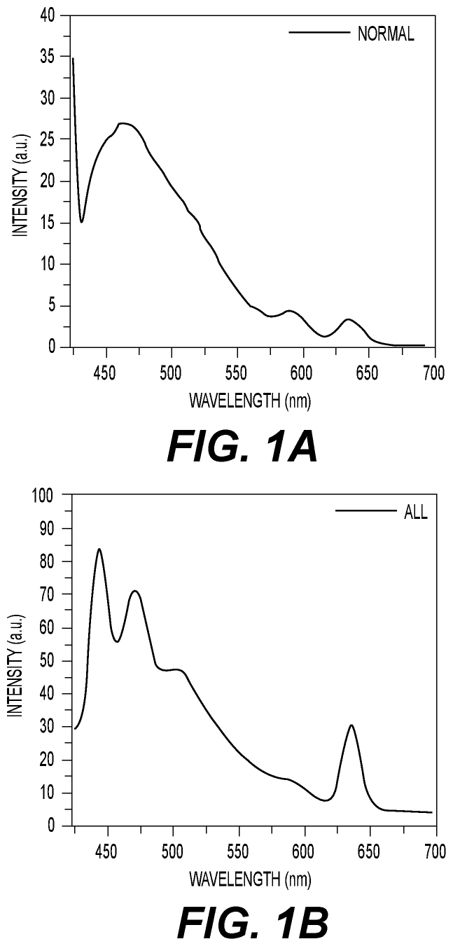

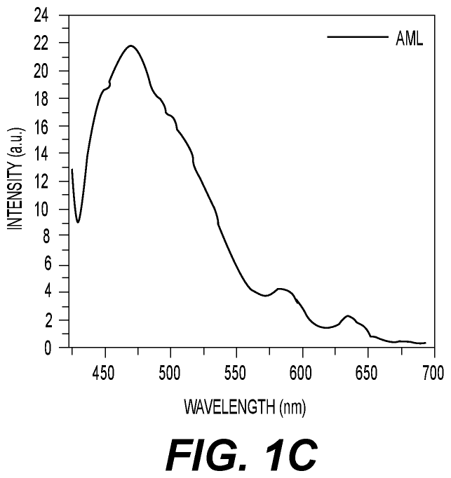

FIG. 1A is the FES of acetone extract of subject of normal control blood; FIG. 1B is the FES of acetone extract of subject of blood infected by ALL; and FIG. 1C is the FES of acetone extract of subject of blood infected by AML. There are three major spectral bands, with peaks at 470, 585, and 630 nm, respectively. Out of this, the bands around 470 to 550 nm are due to the fluorescence of background acetone and residual plasma and are unimportant. But the other two bands, at 585 and 630 nm, are due to two types of porphyrin, an essential protein of RBC. The intensity at 630 nm, which is due to neutral porphyrin, is a measure of oxygen carrying capacity. This is different for both the types of blood cancers.

In order to highlight the distinct differences, a ratio parameter is defined as:

$$R 1 = \frac{intensity at 630 nm}{intensity at 585 nm} = I 630 / I 585$$

i.e., R1=I630/I585 (concentration of neutral to basic porphyrin). This ratio is tabulated below.

TABLE 1
R₁ for normal blood and acute leukemia

| Ratio parameter | Normal | ALL | AML |
|---|---|---|---|
| R₁ = I630/I585 | 1 ± 0.1 | 0.5 ± 0.2 | 0.6 ± 0.05 |

The above ratio parameter was the average of a minimum of 20 samples of each set. The standard deviation in each case is ±10%, yet they do not overlap. Hence, each set can be classified unambiguously

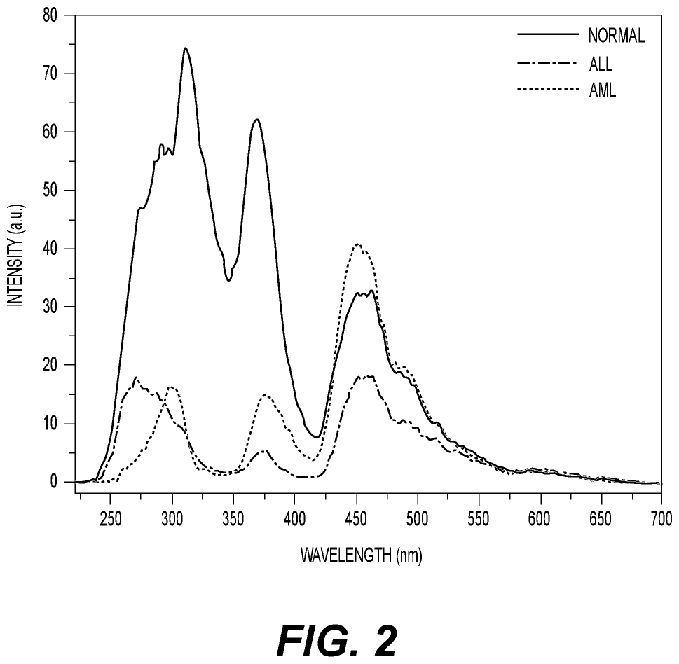

FIG. 2 gives the SFXS of plasma of all three subjects, including spectra for normal blood, for ALL, and for AML. In the above spectra, normalization was not done, since the spectra varied dramatically from one disease to the other (as seen in the Table below). Again in order to highlight the characteristic features, two more important ratio parameters have been defined. They are: R2=I270/I300 (ratio of concentration between amino acid tyrosine and another amino acid tryptophan) and R3=I450/I370 (ratio of concentration of coenzyme FAD and NADH). Here, I270 means the spectral intensity at 270 nm; others follow similar definition.

TABLE 2
Ratio of components by SFXS

| Parameter | Normal | ALL | AML |
|---|---|---|---|
| ′peak | 72 | 14 | 16 |
| R2 = I270/I300 | 0.7 | 1.2 | 0.9 |
| R3 ⁼ I450/I370 | 0.7 | 1.5 | 3.0 |

Here again each ratio parameter varies by ±10% as standard deviation; yet each could be classified individually from each other with 100% accuracy.

The most conspicuous and unmistakable difference between the normal and acute leukemia (ALL and AML) sample is this. The intensity around 290 nm is about 4-fold (400%) less. When a subject is found with this level of drastic reduction then the subject has to be diagnosed with either ALL or AML. When R2 is 1.2, the subject is ALL, and otherwise AML. Similarly, when R3 is 2 times higher than for normal, it is ALL. If 3 times higher, it is AML. This is because ALL has only mild elevation of FAD, an enzyme involved in the redox process, but this is almost tripled in AML.

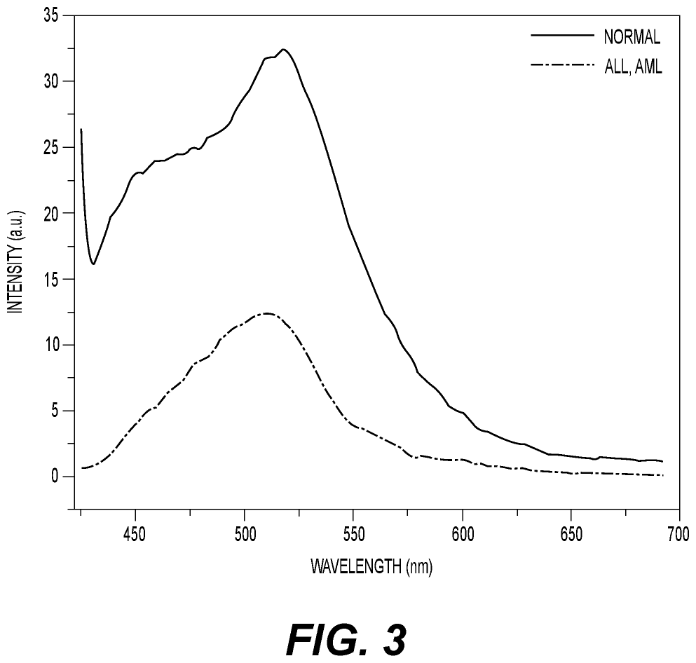

FIG. 3 gives the FES of plasma. It can be seen that the spectral shape and intensity between the spectrum of normal blood and blood infected by acute leukemia (either ALL or AML) is unmistakably different. The intensity is twice as less for AL, and what is very conspicuous is R3=1520/1450 is 1.5 for normal; but 6 for AL (ALL or AML). An important (non-spectroscopic) parameter for the classification of above variants is the ratio between volume of plasma/volume of cellular components.

TABLE 3
R4 values for normal blood and for acute leukemia

| Parameter | Normal | ALL | AML |
|---|---|---|---|
| R4 | 1.2 | 2.5 | 2 |

A professional pathologist and hematologist may visualize the diagnostic protocol for blood cancer in the following lines. A subject complains of night chill (due to weak WBC), excessive bleeding (due to low platelet counts), joint pain (low oxygen content of blood). He is suspected of some blood disorder. In step 1, the subject is advised to give 5 ml of blood, i.v., in an EDTA vial. In step 2, the blood is centrifuged and the volume of plasma and cellular component (CC) measured. If the plasma is 1.7 to 2.3 times of CC the subject may have AL (may be ALL or AML). In step 3, the CC is lysed with acetone, and FES is taken with the spectral detector. If the ratio R1 is only 0.5-0.7, it may indicate general anemia or AML or ALL. In step 4, the plasma is subjected to SFXS with the spectral detector. If the peak intensity is around 70, it is normal, but around 15-30, i.e., 4-fold low, it must be AL (may be ALL or AML). In step 5, the plasma is subjected to FES with the spectral detector. If the intensity ratio R3 is 1-2, normal; but anywhere above 3 it must be AL (may be ALL or AML). In step 6, if the SFXS of plasma shows R2=0.8, and if R3 is 2 to 4, it must be AML and not ALL.

If there is an interfering disease that could mislead diagnosis, it must be beta thalassemia, which is many times more common in countries like Maldives. For beta thalassemia, also if R1=0.6, R2=0.8, and R3=6, this is an indication of hemolysis in the case of Thalassemia.

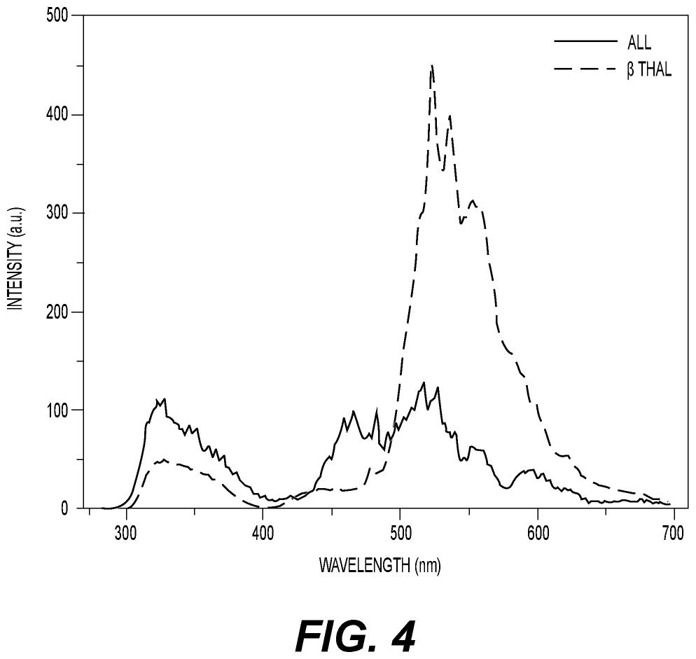

In order to have a much better discrimination, SFES (synchronous fluorescence emission spectra) is taken and is shown for ALL and Bthal. Define a new ratio R5=1555/1460, it is >4 for beta thalassemia (see FIG. 4) and <1.5 for AL (ALL or AML). Thus we have a unique set of biomarker values and algorithm for classifying AML and ALL from all the rest of blood disorders.

We thank, This Project was funded by the National Plan for Science, Technology and Innovation (MAARIFAH), King Abdulaziz City for Science and Technology, Kingdom of Saudi Arabia, Award Number (3-17-05-001-0056).

It is to be understood that the spectrographic detection of acute leukemia is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method for treating acute leukemia, comprising the steps of:
obtaining a sample of blood from a patient exhibiting signs and symptoms of a blood disorder;
extracting cellular components from the blood sample in acetone to lyse cellular components in the blood sample and provide an acetone extract;
obtaining a fluorescence emission spectrum (FES) of the acetone extract;
determining the ratio of intensities in the FES spectrum of the acetone extract at 630 nm to 585 nm (I630/I585);
determining that the patient suffers from either anemia or acute leukemia when the ratio of I630/I585 in the FES spectrum of the acetone extract is between 0.5 and 0.7, and
determining that the patient suffers from acute leukemia by obtaining a synchronous fluorescence excitation spectrum (SFXS) of plasma from the blood sample and determining that either acute lymphoblastic leukemia (ALL) or acute myeloid leukemia (AML) is present in the blood sample when peak intensity in the SFXS spectrum of the plasma from the blood sample is between 15 and 30;
determining the ratio of intensities in the SFXS spectrum of the plasma from the blood sample at 270 nm to 300 nm (I270/I300) and at 450 nm to 370 nm (I450/I370);
determining that the patient suffers from AML when the ratio of intensities in the SFXS spectrum of the plasma from the blood sample at 270 nm to 300 nm (I270/I300) is 0.8 and the ratio of intensities in the SFXS spectrum of the plasma from the blood sample at 450 nm to 370 nm (I450/I370) is 2 to 4;
determining that the patient suffers from ALL when the ratio of intensities in the SFXS spectrum of the plasma from the blood sample at 270 nm to 300 nm (I270/I300) is not 0.8 or the ratio of intensities in the SFXS spectrum of the plasma from the blood sample at 450 nm to 370 nm (I450/I370) is not 2 to 4; and
instituting treatment to the patient for acute leukemia when it is determined that the patient suffers from either ALL or AML.

2. (canceled)

3. The method for treating acute leukemia according to claim 1, wherein determining that the patient suffers from acute leukemia further comprises:
obtaining a fluorescence emission spectrum (FES) of plasma from the blood sample;
determining the ratio of intensities in the FES spectrum of the plasma from the blood sample at 450 nm to 370 nm (I450/I370); and
determining that the patient suffers from either ALL or AML when the ratio of I450/I370 in the FES spectrum of the plasma from the blood sample is greater than 3.

4. (canceled)

5. The method for treating acute leukemia according to claim 1, wherein determining that the patient suffers from acute leukemia further comprises:
obtaining a synchronous fluorescence emission spectrum (SFES) of plasma from the blood sample;
determining the ratio of intensities in the SFES spectrum of the plasma from the blood sample at 555 nm to 460 nm (I555/I460);
ruling out a determination of acute leukemia when the ratio of intensities in the SFES spectrum of the plasma from the blood sample at 555 nm to 460 nm (I555/I460) is more than 4 due to interference from beta thalassemia in the plasma from the blood sample; and
confirming a determination of acute leukemia when the ratio of intensities in the SFES spectrum of the plasma from the blood sample at 555 nm to 460 nm (I555/I460) is less than 1.5.

6. The method for treating acute leukemia according to claim 1, wherein determining that the patient suffers from acute leukemia further comprises:
periodically repeating the method steps during a course of treatment for acute leukemia to determine whether treatment is resulting in improvement or remission of the acute leukemia.
